(19) United States
(12) Patent Application Publication
(10) Pub. No.: US 2007/0031914 A1
(43) Pub. Date: Feb. 8, 2007

(54) Devices for analyte assays and methods of use

(76) Inventors: Wei Zhu, Hangzhou (CN); Xiaoqiang Xu, Hangzhou (CN); Haipeng Hu, Hangzhou (CN)

Correspondence Address: AZURE INSTITUTE;INTELLECTUAL PROPERTY DEPT., 4108 SORRENTO VALLEY BOULEVARD, SAN DIEGO, CA, 92124, US

(21) Appl. No.: 11/198,906

(22) Filed: Aug. 5, 2005

(51) Int. Cl. C12Q 1/26; C12M 1/34

(52) U.S. Cl. 435/25; 435/287.1

(57) ABSTRACT

The present invention provides devices for detecting the presence of an analyte in a liquid sample. The parts of the device include a sample collector for collecting and applying a liquid sample, a test matrix with reagents for detecting the analyte, a sample application port for receiving the liquid sample, the port being in fluid communication with the test matrix, a storage port for storing the sample collector in contact with the device, a window though which at least a portion of the matrix is visible, and a standards card having one or more color standards for comparison with the test matrix. In one embodiment the analyte detected is alcohol is saliva (ethanol). In use the sample collector is placed in contact with the sample (e.g., in the mouth of the subject) and filled with sample, and the sample is expressed from the collector at the sample application port to begin the assay. The standards cards on the device are used for comparison to identify the concentration of analyte in the sample. Methods of use and kits are also provided.

### FIELD OF THE INVENTION

The present invention is directed to devices for the collection of fluid samples and the analysis of the samples for the presence of an analyte.

### BACKGROUND OF THE INVENTION

The following Background of the Invention is intended to aid the reader in understanding the invention and is not admitted to be prior art.

Alcohol and illicit drug use is an established and growing problem in our society. According to the 2001 National Household Survey on Drug Abuse (NHSDA), 46.6% of Americans aged 12 and older reported drinking alcohol in the past 30 days. Approximately 20.5% of persons ages 12 or older participated in binge drinking. Heavy drinking was reported by 5.7% of the population aged 12 or older, or 12.9 million people. In 2001, more than 1 in 10 Americans aged 12 or older (25.1 million persons) drove under the influence of alcohol at least once in the 12 months prior to the interview.

In 2003, the US Department of Health and Human Services found that an estimated 19.5 million Americans or 8.2% of the population aged 12 or older, were current illicit drug users. Current illicit drug use means use of an illicit drug during the month prior to the US Department of Health and Human Services survey interview. Marijuana was found to be the most commonly used illicit drug, with a rate of 6.2% (14.6 million). An estimated 2.3 million persons (1.0%) were current cocaine users, 604,000 of whom used crack. Hallucinogens were used by 1.0 million persons, and there were an estimated 119,000 current heroin users.

Alcohol and illicit drug use has a heavy cost to society. One-quarter of all emergency room admission, one-third of all suicides, and more than half of all homicides and incidents of domestic violence are alcohol-related (Sobering Facts on the Dangers of Alcohol,” NY Newsday, Apr. 24, 2002). Almost half of all traffic fatalities are alcohol-related (National Highway Traffic Safety Administration, Annual Report, 1992). Alcohol and drug abuse costs the American economy and estimated $276 billion per year in lost productivity, health care expenditures, crime, motor vehicle crashes, and other conditions (“Substance Abuse: The Nation's Number One Health Problem,” Institute for Health Policy, Brandeis University, 2001). Untreated addiction is more expensive than heart disease, diabetes, and cancer combined (“Substance Abuse: The Nation's Number One Health Problem,” Institute for Health Policy, Brandeis University, 2001). Every American adult pays nearly $1,000 per year for the damages of addiction (The National Drug Control Strategy, The White House, 1997).

To combat and monitor this problem, drug and alcohol testing has become standard procedure in a variety of settings, such as employment, school, sports, law enforcement, and the like. To facilitate this effort, a drug-testing industry has emerged. This industry provides a variety of drug and alcohol testing products. A common testing product is a urine collection cup incorporating analyte tests. These devices can be complicated and difficult or messy to use, or they may pose special problems of sample adulteration by the subject trying to hide their recent drug or alcohol abuse. In addition, urine samples cannot be collected in certain situations, such as on the road side or in public.

There is therefore a need for better methods and apparatuses for performing sample collection and testing.

### SUMMARY OF THE INVENTION

The present invention provides devices, methods and kits for detecting the presence of an analyte in a liquid sample. In some embodiments, the devices are self-contained assay devices for detecting the presence of alcohol (ethanol) or another analyte in saliva or fluid samples. In some embodiments these devices contain a test matrix having reagents for detecting the presence of ethanol, a sample collector having an absorbent member for collecting a saliva sample, a sample application port for transferring sample from the collector to the device, and a storage port on the device for storing the collector. The devices also can have a standards card attached to the device for comparing the color development of the test matrix to a standard, thereby indicating the concentration of ethanol in saliva (or of another analyte in a fluid sample). In one embodiment the standards card moves slideably on the device to move the standard next to the test matrix and facilitate the comparison. The invention also provides methods of determining the presence or concentration of detecting the presence or concentration of an analyte in a fluid sample (e.g., the concentration of ethanol in saliva). Kits containing the devices and instructions for using the devices are also provided.

In one aspect the present invention provides a test device for detecting the presence or concentration of an analyte in a fluid sample. The test device contains a sample collector for collecting and applying a liquid sample; a test matrix comprising reagents for detecting the analyte; a sample application port for receiving a liquid sample, the port being in fluid communication with the test matrix; a storage port for storing the sample collector in contact with the device; a window through which at least a portion of the matrix is visible; and a standards card having one or more color standards for comparison with the test matrix.

In one embodiment the sample collector of the device has an absorbent member situated on a protruding stick. The absorbent member can be made of a porous material such as a sponge, or another porous material that can absorb and hold a liquid sample for transferring to the device. The sample collector can also have a dental support to facilitate the subject holding the collector in the mouth (when saliva is the liquid sample of interest). In a further embodiment, the application port has an expression surface for separating the sample from the sample collector.

In another embodiment the test matrix exhibits a first color before the sample is applied to the application port and reacts with the reagents. After the sample has been applied to the application port, the test matrix changes to a second color. The test matrix changes to the second color after the analyte has reacted with the reagents. The second color indicates the concentration of the analyte in the sample. In further embodiments, the reagents are color-changing reagents. In one embodiment the test matrix contains reagents for determining the presence or concentration of ethanol in saliva.

In another embodiment, the standards card is slideably moveable on or within the device to place one or more color standards adjacent to the text matrix. In a further embodiment, the storage port includes a tube into which the sample applicator is secured. The sample application port may be located at the end of the tube. The fluid sample can be whole blood, a blood product, urine, saliva, cerebrospinal fluid, tears, a vaginal swap, a throat swab, or a nasal swab. In certain embodiments, the fluid sample is saliva.

In various embodiments, the analyte can be a drug of abuse or a metabolite thereof. In one embodiment, the analyte is ethanol and the reagents comprise alcohol oxidase, horseradish peroxidase and tetramethylbenzidine. In another embodiment, the analyte is ethanol and the reagents comprise alcohol dehydrogenase, nicotinamide adenine dinucleotide, NADH diaphorase and a tetrazolium dye.

In another aspect the present invention provides methods for determining the presence or concentration of an analyte in a fluid sample. In one embodiment, the methods involve applying a fluid sample from an absorbent member of a sample collector to a device of the invention as described herein, moving the standards card to a position so that the standard is adjacent to the test matrix; and comparing the color of the test matrix to a standard to determine the concentration of analyte in the fluid sample. In certain embodiments, the fluid sample is saliva.

The matrix changes from a first color to a second color when the reagents react with the analyte, and the second color is indicative of the concentration of the analyte. In a further embodiment, the step of moving the standards card includes sliding the standards card linearly through the device so that the standard is adjacent to the test matrix.

In another embodiment, the step of applying the sample fluid includes pressing the absorbent member of the sample collector against an expression surface on the device. In this embodiment, the expression surface is located in the sample application port. In certain embodiments of the present method, the analyte is a drug of abuse or ethanol.

In another aspect, the present invention provides kits for detecting an analyte in a fluid sample. In one embodiment the kit includes a test device of the present invention packaged together with instructions for use of the device. In a further embodiment, the instructions describe the use of the device to detect ethanol in saliva.

The summary of the invention described above is not limiting and other features and advantages of the invention will be apparent from the following detailed description, as well as from the claims.

### BRIEF DESCRIPTION OF THE DRAWINGS

FIG. 1 provides a perspective view of the device of the present invention 100.

FIG. 2 provides an exploded view of the device shown in FIG. 1.

FIG. 3 is a cut-away and enlarged view of the device illustrated in FIG. 1.

FIG. 4 is a cut-away perspective view of the device illustrated in FIG. 1, illustrating insertion of the sample applicator 110 into the storage port 312 via the application port 410.

FIG. 5 illustrates expression of a collected sample from the absorbent member 210 by manually pressing the absorbent member against the expression surface 310.

FIG. 6 illustrating sliding the standards card 118 within the standards groove 220 to compare the intensity of the color of the test matrix 114 to the colors of the standards 218.

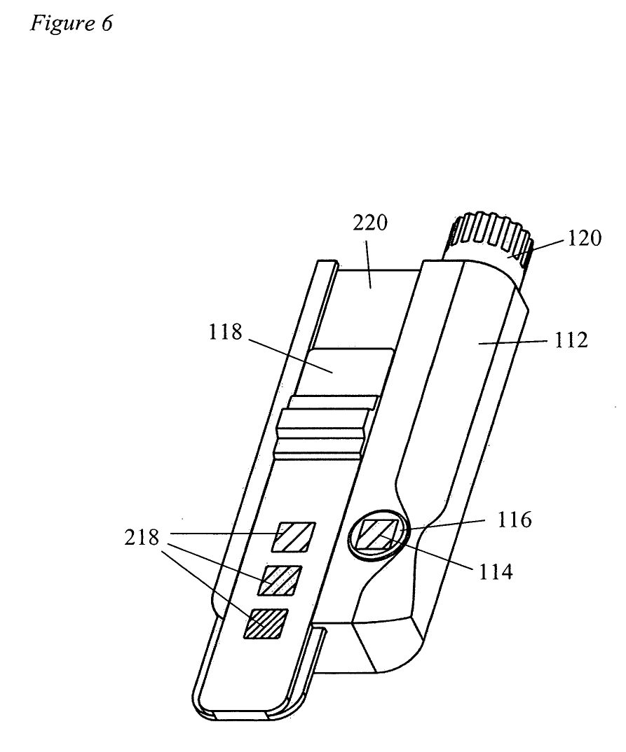

### DETAILED DESCRIPTION

In the following detailed description, reference is made to the accompanying drawings that form a part hereof, and in which is shown by way of illustration specific embodiments in which the invention may be practiced. It is understood that other embodiments may be utilized and structural changes may be made without departing from the scope of the present invention.

Test Devices

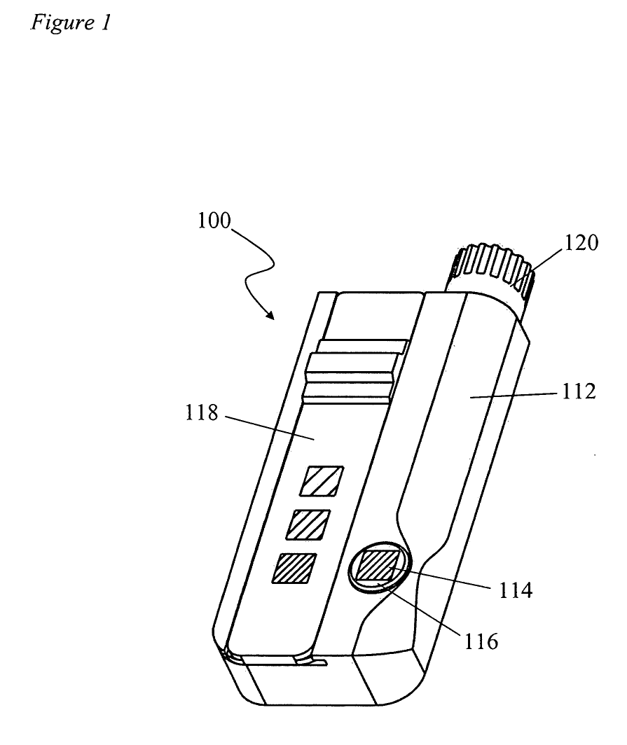

With reference to the Figures, the invention provides a test device 100 including a sample collector 110, a test matrix 114, a sample application port 410, a window 216, and a standards card 118. The test device can also include a housing 112. One embodiment of the present device is illustrated in FIGS. 1 though 6. It will be readily understood by those in the art that various configurations of the present device can be made, and those are also within the scope of the present invention.

Sample Collector

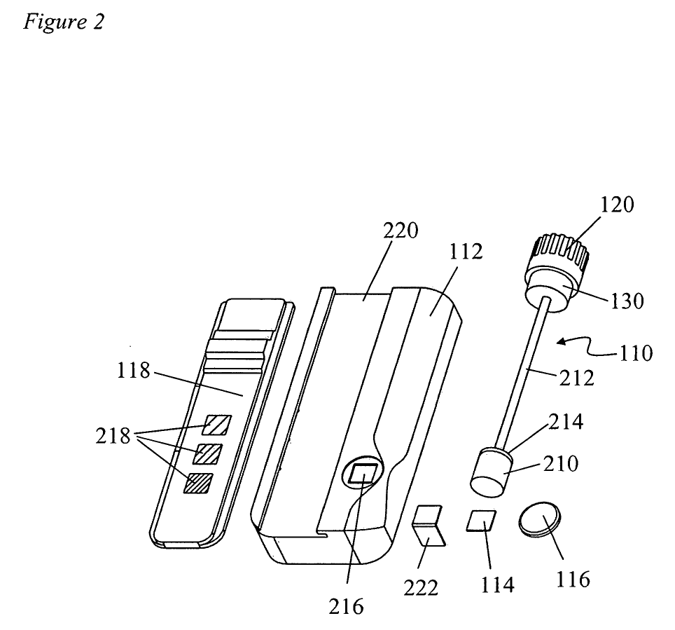

In one embodiment, the device of the invention contains a sample collector 110 for collecting sample fluid. With reference to FIG. 2, the sample collector 110 has an absorbent member 210 attached to one end of a stick 212 for supporting the absorbent member. The sample collector can also have a handle 120 for grasping and manipulating the sample collector, and a lower handle 130. In some embodiments the lower handle 130 is configured for securing the collector in a storage port on the device. The lower handle 130 can fit securely into a structure on the device, into which it is secured by mechanical pressure. For example, the lower handle 130 can contain a ring of rubber or other material and the handle can be sized so that it fits securely into an opening on the device due to mechanical pressure of the rubber ring fitting into the opening for the collector. The absorbent member can be made of any porous material that absorbs fluid and that can withstand the mechanical stress of being manipulated in the assay. When the sample fluid is saliva, the absorbent member is made of a porous and non-toxic material that can withstand the mechanical stress of being manipulated within the mouth during sample collection. In one embodiment, the absorbent member is made of medical grade sponge or closed-cell foam, which is available from various sources (e.g., Avitar, Inc. Canton, Mass., USA). In one embodiment the material of the absorbent member expands in size as it is contacted and filled with the saliva sample. In other embodiments the absorbent member can be made of absorbent paper, cotton, or any material that holds fluid and that can transmit the fluid from the mouth to the sample application port of the device. The absorbent member can also be treated with agents that stimulate salivation, for example, flavorings or a dilute citric acid solution. The term “saliva” refers to the secretions of the salivary glands.

When the fluid sample is saliva, the stick can include a dental support 214, which can provide a surface for attachment of the absorbent member, as well as a convenient structure that is easily held within the mouth. The dental support can comprise a ring or circle of plastic affixed to the sample collector, which can then be comfortably supported behind the teeth of the subject using the dental support. The stick and dental support of the sample collector are conveniently made of a plastic material. For example, polypropylene, polystyrene, and polycarbonate are all examples of suitable plastics. Additionally, these components can be made of wood or any material having the qualities and abilities to serve in the assay as described herein. The absorbent member can be attached to either the dental support or the end of the stick by any convenient means (e.g., by a medical grade oral adhesive). Suitable medical adhesives (e.g., UV-curing adhesives) are commercially available from various sources (e.g., DYMAX Corp., Torrington, Conn. USA). The dental support can be of any shape, such as a circular or oval shape, a square shape, or any shape that is easily grasped by the teeth and other structures of the mouth.

Test Matrix

The present device includes a test matrix 114, which comprises reagents for detecting an analyte in the sample. Any material that absorbs and transports a fluid sample can be used as the test matrix. The fluid sample can be transported within the test matrix by capillary action, meaning movement through the test matrix by capillary forces. In various embodiments the test matrix is a bibulous material that transports liquid by capillary action and does not interfere with the reaction between the reagents and the analyte being detected. In one embodiment the matrix material is a polyamide fiber membrane. The matrix can have any appropriate thickness, e.g., from 0.6 to 1.0 mm, or from 0.8 to 1.2 mm, or from 0.8 to 1.0 mm, or from 0.9 to 1.1 mm. A 60 mm×10 mm piece of polyamide fiber material of appropriate thickness will absorb 0.6 gm of fluid, ±0.15 gm. Such polyamide fibers are available from a variety of commercial sources (e.g., Filtrona Fibertec™ Colonial Heights, Va.). Of course many other bibulous materials can also be useful as the test matrix. For example, surface active media that utilize either amine or carboxyl groups on the surface of the fiber as substrates for a wide variety of linking agents also functions well in the present invention and is available from a variety of commercial sources (e.g., Filtrona Fibertec™). In other embodiments, cotton fiber or polyester can be used as the test matrix. These materials can be advantageously treated with detergents, proteins, and buffers, depending on the specific application involved. In some other embodiments the test matrix can be made of nylon fiber, nitrocellulose, polyester fibers, cellulose esters, and cellulose acetates (e.g., cellulose triacetate). With reference to the present disclosure the person of ordinary skill in the art will realize various other materials that will find use in the present invention as a test matrix.

Test reagents can be applied to the test matrix by any convenient means, for example by soaking the test matrix material in a solution of the reagents followed by drying. In other embodiments reagents can be applied to the matrix by spraying, application with a dropper, or applied to the matrix by any other means and allowed to dry. At least some of the test reagents react with the analyte or a bi-product of the analyte (if present in the sample) to produce a color on the test matrix. Thus the test matrix changes from a first color to a second color when analyte is present in the sample. The reagents can be modified depending upon the analyte of interest and the specific application involved. Many chemical and enzymatic tests for blood chemistry or environmental chemistry can be utilized in devices of the invention. In various embodiments reagents can be selected to detect or determine the concentration of ethanol in saliva, the glucose concentration in blood or urine, lead content in household paint, the concentration of an analyte in a water sample, and many other applications. Additionally, the reagents can be modified to accommodate the type of fluid sample used, such as saliva, blood, urine, or water.

In one embodiment, when a target analyte or a bi-product of the analyte is present in the fluid sample, it reacts with reagents contained on the test matrix and causes the color of the test matrix to change from a first color to a second color. The intensity of the second color can correlate with the concentration of the analyte in the applied sample. For example, prior to sample application the matrix may be white. In this embodiment the matrix turns to a blue color after the sample has been applied and has reacted with the analyte (or a bi-product thereof). The concentration of analyte in the sample correlates with the intensity of the blue, from light blue (low analyte concentration), to medium blue, to dark blue (high analyte concentration). Intermediate blue shades can also correlate with corresponding intermediate analyte concentrations. Any color can be used as a first color or second color (e.g., red, orange, yellow, etc.) as long as the first color indicates no analyte present and the second color indicates the presence or concentration of analyte (or a bi-product thereof), and that the second color has a sufficient number of discemable shades that correlate with different concentrations of the analyte. The first and second colors of the matrix are dependent upon the reaction components selected for a particular application. By “bi-product” of an analyte is meant a molecule other than the target analyte itself, the presence or concentration of which is directly related to the presence or concentration of the analyte. For example, the metabolic product of an analyte can be a bi-product of the analyte.

In certain embodiments, the test matrix may be provided in a housing 112 having a window 216. One embodiment of a housing is illustrated in the Figures. At least a portion of the matrix is visible through the window. The window can be an opening in the housing through which the test matrix can be viewed within the housing. The opening may be covered or uncovered. But in other embodiments the window may be on the exterior of the housing, and on which the matrix can be supported for viewing by the device user. In further embodiments, the window has a clear cover 116, through which the test matrix can be seen. The window cover protects the test matrix from exposure to the exterior environment and during handling of the device.

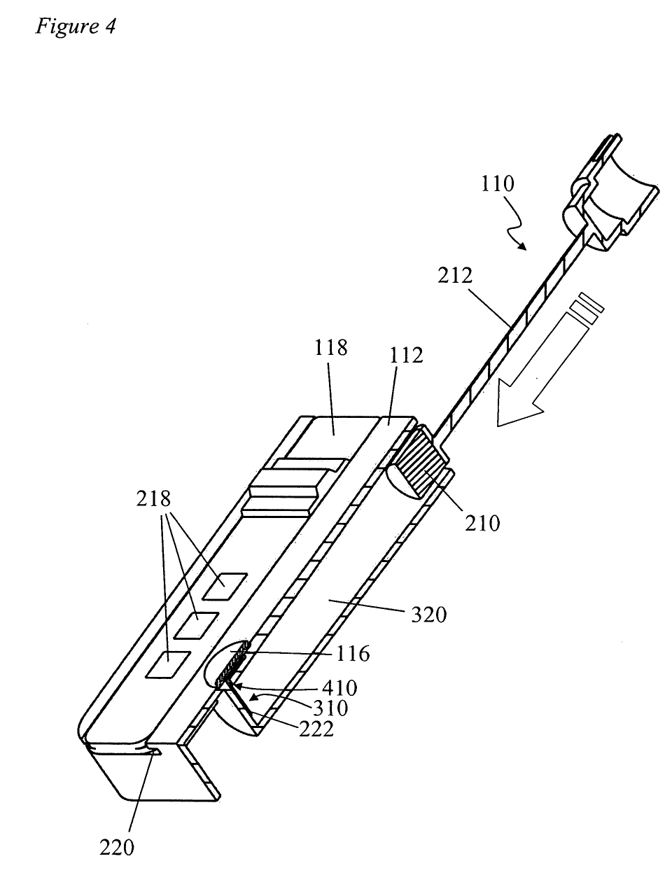

In some embodiments the device has a sample application port for receiving the liquid sample in the device. The sample application port allows communication between the sample collector and the test matrix, through the device. The sample application port 410 is the area of the device where sample is applied. With reference to FIG. 4, the sample application port 410 is an area or opening located at the bottom of a well or tube on the device, but can also be located directly on an exterior surface of the device. The well or tube can also be useful for holding and storing the sample collector prior to and after use. But the sample application port can be present in a variety of forms. For example, in one embodiment the sample application port is an opening that leads through a passageway or channel to the test matrix. Sample expressed at or near the application port passes through the opening and into the test matrix. In certain embodiments, the window can also serve as the application port. In addition to applying the sample with the sample collector, the sample may be applied to the matrix by other convenient means, such as with a dropper or pipette.

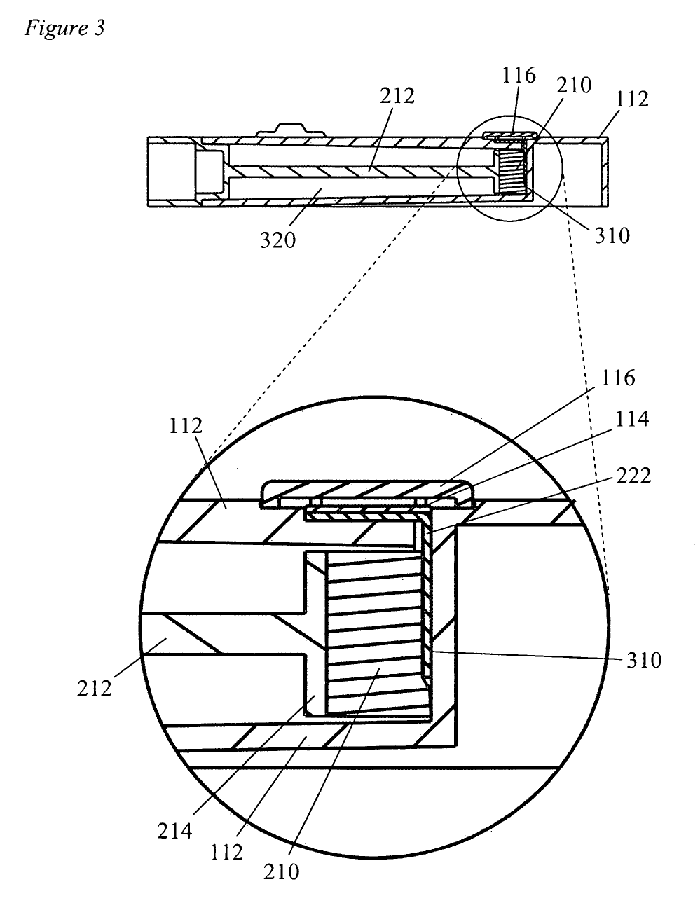

In one embodiment, the application port has an expression surface 310 that is located so that expressed sample is transferred to the test matrix. The application port is thus in fluid communication with the test matrix (see FIG. 3). When the applicator is pressed against the expression surface, a portion of the saliva is expressed from the absorbent member. With reference to FIGS. 3 and 4, in some embodiments the sample application port is in fluid communication with the test matrix through a passageway or channel. When fluid sample is expressed at the sample application port, it flows through the passageway or channel and is absorbed by the test matrix, thereby initiating the assay. An absorbent material 222 is present in the channel and transmits the fluid sample from the application port to the test matrix (e.g., by a wicking action or capillary action). The absorbent material can be filter paper or another absorbent material that effectively transfers liquid. With reference to FIG. 4, in one embodiment the absorbent material is bent at an angle (e.g., a 90° angle ±10°) so that the material places the expression surface into fluid communication with the test matrix. The absorbent material transfers sample expressed from the sample collector at the sample application port to the test matrix. Any bibulous material that readily transmits fluid (e.g., by wicking or capillary action) can be used as the absorbent material. In some embodiments, the absorbent material is treated to prevent adsorption of the analyte to the absorbent material. For example, when the absorbent material is filter paper, it can be treated with buffers, surfactants and nonspecific proteins to reduce the adsorption of drug analytes to the paper. In some embodiments the sample application port has a specialized expression surface, such as an expression plate. The plate can take the form of a disc (and may optionally have holes in it to allow the passage of sample fluid) that is situated so that expressed fluid is transferred to the test matrix. In other embodiments the application port does not have a separate expression surface, and the filled sample collector can simply be pressed against or near the application port to express sample. In another embodiment the filled sample collector can be pressed directly against the absorbent material, which can be present on the surface of the sample application port. By “fluid communication” is meant that a quantity of fluid sufficient to conduct the assay can flow from one structure to another when the two structures are in fluid communication. By “expression surface” is meant any surface of sufficient strength, area, and rigidity to allow sample fluid to be extracted from the absorbent member by mechanical pressure of the absorbent member against the expression surface. In one embodiment the expression surface has an area at least equivalent to the area of one side of the absorbent member.

In other embodiments, the device includes a storage port 320, for storing the sample collector. In the embodiment shown in FIG. 4, the storage port 320 is present in the form of a tube or column situated on the device. The sample collector fits within the tube and is thereby conveniently protected from contamination or mechanical damage. Of course embodiments in which the storage port has another configuration are also possible and within the scope of the present invention. For example, in one embodiment the storage port can be present as a clip on the side of the device and into which the sample collector snaps to remain securely fastened to the device. In another embodiment the sample collector is stored separately from the device. In one embodiment the sample collector contains a small rubber gasket or ring that fits around the lower handle 120 of the collector. The gasket can fit snugly into a structure on the storage port (e.g., its opening) to secure the sample collector in a stable position.

In one embodiment the device has a window through which at least a portion of the test matrix is visible. The window can have a piece of glass, plastic, or another transparent substance to protect the test matrix from contamination. In another embodiment the window can be simply an opening in the device that allows the user to view the test matrix.

In some embodiments the device has at least one standards card 118 for correlating the second color of the test matrix with the presence of analyte (or a bi-product thereof) with a specific analyte concentration indicated on the standards card. The standards card carries at least one colored standard 218 indicating a corresponding concentration for the individual colors. In the illustrative embodiment shown in the Figures, the device has a structure, such as a groove 220 into which the standards card is slideably present. In the embodiment shown in the Figures, the standards card is sized and shaped to mate with the groove and to be slideably moveable therein. The standards card slides within the groove, allowing the colored standards to be aligned next to the test matrix for enhancing the ease of color comparison between the standards card and the test matrix. In one embodiment the standards card is slideable on the device in the elongate direction, meaning parallel to direction of the stick of the sample collector when the collector is in the stored position. In other embodiments the standards card can be glued or otherwise fixed on the device. The cards can also be present on a wheel that can be turned to align a standard next to the test matrix for easy comparison. The standards card can also be provided detached from the device. In another embodiment the standards are provided individually and separate from the device, and the device contains a receiving area to receive an individual standard card in proximity to the test matrix for easy comparison. By “slideable” or “slidably” is meant that the standards card is fitted into a groove on the device and that the card can be slid within the groove to change the standard that is located closest to the test matrix.

Analytes

A variety of well known calorimetric, enzymatic, and chemical tests can be adapted for use with the device of the present invention. Any analyte of interest can be detected or its concentration in a fluid determined using the presence invention, as long as there exists a test that can be incorporated into the test matrix. In various embodiments the device can be used to assay for the presence or amount of alcohol in saliva, to determine blood chemistry values (e.g., pH, creatinine, glucose, alkaline phosphatase, etc.), the presence or amount of alkaline phosphatase in milk, and ammonium, phosphate, lead, or arsenic in ground water. The analyte can also be a bi-product of the analyte of interest. Thus, in those cases where the analyte is unstable or for another reason is not preferred as the actual analyte identified, a more convenient bi-product can be the actual analyte identified, which correlates with the presence or concentration of analyte in the sample.

In one embodiment the analyte is a drug of abuse or a bi-product thereof. A “drug of abuse” (DOA) is a drug that is taken by a subject for non-medicinal reasons (usually for mind-altering effects). The abuse of such drugs can lead to physical and mental damage and (with some substances) dependence, addiction and/or death. Some drugs of abuse act on the central nervous system. Examples of DOAs include cocaine; amphetamines (e.g., black beauties, white bennies, dextroamphetamines, dexies, beans); methamphetamines (crank, meth, crystal, speed); barbiturates (e.g., Valium®, Roche Pharmaceuticals, Nutley, N.J.); sedatives (i.e. sleep-aids); lysergic acid diethylamide (LSD); depressants (downers, goofballs, barbs, blue devils, yellow jackets, ludes); tricyclic antidepressants (TCA, e.g., imipramine, amitriptyline and doxepin); phencyclidine (PCP); tetrahydrocannabinol (THC, pot, dope, hash, weed, etc.); and opiates (e.g., morphine, opium, codeine, heroin, oxycodone).

Reagents

In one embodiment the reagents used in the invention are color-changing reagents. In one embodiment alcohol concentration is determined by a reaction of alcohol dehydrogenase (ADH), nicotinamide adenine dinucleotide (NAD/NADH), NADH diaphorase and a tetrazolium salt (which produces a highly colored, purple formazan dye in the presence of alcohol). In another example embodiment, alcohol concentration is determined by a reaction of tetramethylbenzidine (TMB), Alcohol Oxidase and Peroxidase (to produce a blue or green color in the presence of alcohol). With reference to the present disclosure the person of ordinary skill in the art will realize many other chemistry formats that can be used with the present device to determine the presence or amount of many different analytes in many different sample fluids. By “color-changing reagents” is meant reagents that provide a detectable appearance of color or change in color due to the presence of an analyte of interest or a bi-product of an analyte of interest. The color is apparent to the unaided eye and can be determined without the aid of analytical instruments.

In another example, glucose can be detected with a test matrix treated with glucose oxidase, horseradish peroxidase, and either 3-methly-2-benzothiazolinone hydrazone hydrochloride combined with 3,3-dimethylaminobenzoic acid (MBTH-DMAB) or 8-anilino-1-naphthalene sulfonic acid ammonium (MBTH-ANS). Glucose oxidase and horseradish peroxidase can also be combined with [3-methyl-2-benzothiazolinone]-N-sulfonyl benzenesulfonate monosodium and 8-anilino-1-naphthalene sulfonic acid ammonium (MBTHSB-ANS) to detect glucose concentration. Examples of other metabolic indicators include, but are not limited to, creatinine (e.g., see U.S. Pat. No. 4,529,708 to Stephens), chloride (e.g., U.S. Pat. No. 4,393,142 to Stephens), xanthine oxidase (e.g., U.S. Pat. No. 4,341,868 to Nakanishi) and lactic acid (e.g., U.S. Pat. No. 4,266,022 to Lamprecht and U.S. Pat. No. 4,254,222 to Owen).

In yet another example, arsenic may be detected in ground water by using a reaction matrix treated with zinc powder, an acid, an oxidizing agent and mercury (II) bromide. This reaction produces a yellow-brown color in the presence of arsenic.

In a further example, certain drugs of abuse, such as THC concentration, and pH can be detected with the chemical assays, instead of enzymatic assays. See U.S. Pat. No. 5,738,634 to Caillouette as an illustrative example.

Sample Types

Any type of liquid specimen may be analyzed with the present device, including fluid biological specimens collected from patients. Alternatively, fluids derived from other types of specimens dissolved in an appropriate liquid, such as a buffer or water, can also be analyzed. For example, the specimen may be composed of fine powdery materials such as talc, carbon black, pharmaceutical preparations, or gases such as argon or methane. Additional specimens can include atmospheric specimens that can be assayed for particulates or radioactive isotopes such as radon.

In one embodiment the specimen to be tested is a biological specimen, for example a sample from a subject such as an animal or a human. The sample can be any type appropriate for analysis, such as a sample of fluid, tissue, organ or a combination thereof. The biological specimen can also be a sample of other biological material, such as plants, bacteria, cell or tissue cultures, viruses and prions, or food, including food such as material derived from plants or animals or combinations thereof. The sample can be processed prior to introduction into the assay device. In the alternative, a sample and reagent can be combined within a specimen collection container. Such reagents can be used to process a sample, such as digesting solid samples with appropriate reagents such as chemicals, such as acids or bases, or with enzymes such as proteases. Other reagents can be used to extract analytes from a sample, such as extraction of antigens from biological entities, such as antigens from etiological agents such as bacteria, parasites, viruses or prions such as known in the art.

The sample collected by the present device is any material to be assayed for the presence and/or concentration of an analyte in a sample or specimen that can be absorbed by the absorbent member. In one embodiment the sample is a liquid sample. Examples of liquid samples that may be collected using a device of the present invention include bodily fluids including blood, serum, plasma, saliva, oral fluid, urine, ocular fluid, semen, and spinal fluid; water samples, such as samples of water from oceans, seas, lakes, rivers, and the like, or samples from home, municipal, or industrial water sources, runoff water or sewage samples; and food samples, such as milk or wine. Viscous liquid, semi-solid, or solid specimens may be used to create liquid solutions, eluates, suspensions, or extracts that can be samples. For example, throat or genital swabs may be suspended in a liquid solution to make a sample. Samples can include a combination of liquids, solids, gasses, or any combination thereof, as, for example a suspension of cells in a buffer or solution.

Liquid samples can be made from solid, semisolid or highly viscous materials, such as soils, fecal matter, tissues, organs, biological fluids or other samples that are not fluid in nature. For example, these solid or semi-solid samples can be mixed with an appropriate solution, such as a buffer, such as a diluent or extraction buffer. The sample can be macerated, frozen and thawed, or otherwise extracted to form a fluid sample. Residual particulates can be removed or reduced using conventional methods, such as filtration or centrifugation.

Methods of Use

In use, the absorbent member of the sample collector absorbs liquid sample so that the sample collector contains a sufficient quantity of sample to perform the assay. For example, when the test subject is a human and the test sample is saliva, the absorbent member of the collector may be placed into the subject's mouth. As the absorbent member of the sample collector becomes saturated with saliva, it may expand in size depending on the material used as the absorbent member. When urine is the test sample, the sample can be collected by any convenient means, such as by simply be held in the stream of urine until saturated, or by using a clean-catch urine cup and then dipping the absorbent member into the collected urine. When the sample fluid is blood, an aliquot of blood may be collected by dipping the collector into the blood sample. In other settings, the method of collecting the sample will vary, depending upon the sample type. For example, if stream water is to be tested for contamination, the absorbent member can be held directly in the stream or the water collected in a bottle, into which the absorbent member is dipped.

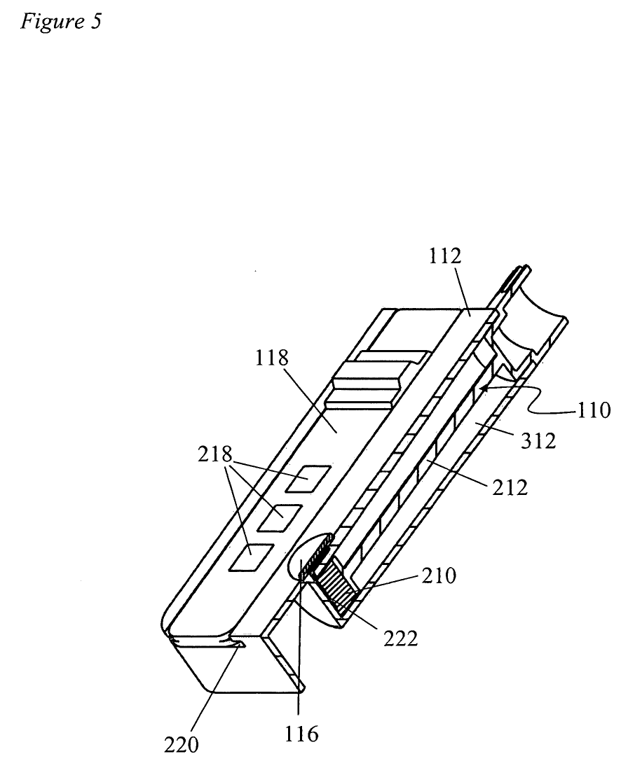

After the sample has been absorbed by the absorbent member, the sample is transferred to the sample application port of the device. Referring to the embodiment of FIGS. 4 and 5, the saturated sample collector is pressed against the expression surface located at the bottom of the storage port to allow sample to enter the sample application port, and be transferred to the test matrix. In some embodiments, the sample collector is inserted into the storage port and the absorbent member is pressed against an expression surface. In the embodiment depicted in FIGS. 4 and 5, an expression surface is present as a bottom wall of the storage port. In various other embodiments, the expression surface can be a plastic or other hard surface at the sample application port. In some embodiments the expression surface has holes in it to allow fluid sample to pass through it and to the sample application port and test matrix.

The test matrix absorbs the liquid sample expressed by the absorbent member. In the embodiment shown in the Figures, the expressed sample is absorbed by absorbent paper, which wicks the sample through a passageway or channel and into the test matrix. When sample is applied to the test matrix, the test reagents present on the matrix react with analyte (if present). If analyte is not present in the sample, the test matrix remains the first color. However, if analyte is present in the sample, reaction of the analyte with the reagents causes the test matrix to turn to a second color. For example, prior to use, the test matrix may be a first color of light yellow. After the test matrix is contacted with the sample, the test matrix will either remain light yellow indicating that no analyte is present, or turn to a second color red, indicating the presence of analyte in the sample. The first and second colors of the matrix depend upon the reagents selected for the reaction. Any colorimetric reaction can be adapted to the device of the present invention, as long as the first and second colors can be visualized and distinguished, and the intensity of the second color can be correlated with analyte concentration.

To interpret the result of the assay, the standards card is moved so that the color of the standards can be easily compared with the test matrix. In one embodiment the standards card is moved linearly through the device so that the standard is adjacent to the test matrix (e.g., by sliding the standards card within a groove on the device). By “adjacent to” is meant the standard is placed so that it is closer to the test matrix than any other standard. By “linearly” is meant moving in a straight line along an axis. Referring to the embodiment depicted in FIG. 1, the user slides the standards card within the groove to determine the most similar standard. The color on the standards card correlates to presence of the analyte or to a specific analyte concentration or range of concentrations which is indicated as being the concentration of anlayte in the test sample.

Kits

The present invention also provides kits for analyzing the presence or concentration of an analyte in a sample. The kits contain at least one device of the present invention provided in a package. The kit can also include instructions for use of the device in the determination of the presence or concentration of an analyte in a test sample. In one embodiment, the device is packaged as a single test device with the instructions for use. In different embodiments, the device can be packaged with or without a sample collector. The format of the kit can vary according to the type of test, the type of sample, and the test environment. In various other embodiments, the kits contains one, two, three, or more than three (e.g., 5, 10, 15 or 20) devices with the instructions for use, provided in a package. In different embodiments the package is a box, a plastic wrapping that envelopes the components, or both. The devices can be individually wrapped, or boxed together with a single set of instructions. In one embodiment the instructions explain the use of the device to detect the presence or concentration of ethanol in saliva.

### EXAMPLE 1

### Assembly of an Alcohol Test Device

This example describes the assembly of one embodiment of the present device, an device for detecting alcohol in saliva, shown in the Figures.

In this example of the invention, the housing and sliding standards card are separately injection molded from rigid plastic. The sample collector stick with dental support is injection molded from a resilient but slightly flexible plastic. Any medical grade plastic can be used for manufacture of the sample collector as long as it provides sufficient support for the absorbent member to be pressed against the expression surface, while at the same time being flexible enough that the stick will bend slightly in the patient's mouth so that the possibility of injury to the patient by the sample collector is minimized. The window cover is injection or press molded of clear plastic.

To make the test matrix, 1.0 mm polyamide fiber material from Filtrona Fibertec™ (Colonial Heights, Va.) is soaked in a reagent solution containing reagents for detecting alcohol (ethanol) in saliva. The solution contains alcohol dehydrogenase (ADH), nicotinamide adenine dinucleotide (NAD/NADH), NADH diaphorase and tetrazolium salt, such as 3-(4,5-dimethylthiazol-2-yl)-2,5-diphenyl tetrazolium bromide, and buffer. After the fiber material is soaked in the reagent solution, it is dried and cut to size. Absorbent paper, such as Whatman 3MM paper (available from Whatman, Inc., Florham Park, N.J., USA), is also cut to size.

A strip of absorbent paper is placed into the passageway connecting the sample application port with the test matrix, so that the sample application port is placed into liquid communication with the test matrix. A test matrix that is cut to appropriate size is placed on top of the absorbent paper and a clear plastic window is placed over the test matrix, so that the test matrix is visible through the window cover. A sticker printed with the standards and corresponding indicia is cut to size and adhered to the standards card. The standards card is then slid into the groove of the housing.

An absorbent member for the sample collector is prepared from hydrophilic medical grade foam, e.g., polyurethane foam. The foam is soaked in a dilute solution of citric acid and then air dried. Round disks, sized to fit onto the dental support of the sample applicator, are cut from the larger sheet of treated foam. A disk is glued to the end of the dental support using medical grade adhesive. The completed sample collector is inserted into the housing storage port. The device is then hermetically sealed in a plasticized foil pouch.

### EXAMPLE 2

### Testing for Alcohol in a Saliva Sample

This example describes laboratory testing of saliva samples having various alcohol concentrations using a device of the invention.

Alcohol-free saliva was collected from normal, healthy volunteers by spitting into a cup. The collected saliva samples were pooled. Four aliquots of the pooled saliva were spiked with ethanol to give final concentrations of 0.00%, 0.02%, 0.040% and 0.08% ethanol. Each concentration of alcohol was tested in replicates of three, using alcohol test devices of the present invention configured for detection of alcohol in saliva. To conduct each test, the absorbent member of the sample applicator was dipped into the prepared sample fluids and allowed to remain submerged for 2 minutes. The sample applicator was then inserted fully into the sample application port and pressed against bottom of the port to express the liquid sample. Expressed sample was absorbed by the absorbent paper and transmitted to the test matrix. After 2 minutes, the test matrix was observed for a change of color, from pale yellow to blue. The standards card was slid next to the window to facilitate comparison of the color of the test matrix with the color of the standard card. The color of the test matrix was matched to the closes color of the standards card and the alcohol concentration of the sample was determined.

The results were as follows: at the 0.00% ethanol concentration, the test matrix remained the initial pale yellow color. Thus, no alcohol was detected in these samples. For the 0.02% alcohol aliquots, the test matrix was changed to a light blue color that matched the 0.02% alcohol standard on the standards card. The test matrices of the 0.04% alcohol test devices turned a medium blue that matched the 0.04% alcohol standard. Similarly, the 0.08% alcohol concentration tests resulted in dark blue matrices, which correlated with the 0.08% saliva alcohol concentration standard on the standards card.

The invention illustratively described herein may be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by various embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

1. A test device for detecting the presence or concentration of an analyte in a fluid sample comprising:
a sample collector for collecting and applying a liquid sample;
a test matrix comprising reagents for detecting the analyte;
a sample application port for receiving a liquid sample, the port being in fluid communication with the test matrix;
a storage port for storing the sample collector in contact with the device;
a window though which at least a portion of the matrix is visible; and
a standards card having one or more color standards for comparison with the test matrix.

2. The device of claim 1 wherein the sample collector comprises an absorbent member situated on a protruding stick.

3. The device of claim 2 wherein the sample collector further comprises a dental support.

4. The device of claim 2 wherein the sample application port comprises an expression surface for separating the sample from the sample collector.

5. The device of claim 4 wherein the sample application port is in fluid communication with the test matrix.

6. The device of claim 1 wherein the test matrix is a first color before application of the sample to the application port, and changes to a second color after the analyte has reacted with the reagents to indicate the concentration of the analyte in the sample.

7. The device of claim 1 wherein the reagents are color-changing reagents.

8. The device of claim 1 wherein the standards card is slideably moveable on or within the device to place one or more color standards adjacent to the text matrix.

9. The device of claim 1 wherein the analyte is selected from the group consisting of:
whole blood, a blood product, urine, saliva, cerebrospinal fluid, tears, a vaginal swap, a throat swab, and a nasal swab.

10. The device of claim 9 wherein the fluid sample is saliva.

11. The device of claim 1 wherein the storage port comprises a tube on the device into which the sample applicator is secured.

12. The device of claim 11 wherein the sample application port is located at the end of the tube.

13. The device of claim 1 wherein the analyte is a drug of abuse or a bi-product thereof.

14. The device of claim 1 wherein the test matrix contains reagents for determining the presence or concentration of ethanol.

15. The device of claim 1 wherein the analyte is ethanol and the reagents comprise alcohol oxidase, horseradish peroxidase and tetramethylbenzidine.

16. The device of claim 1 wherein the analyte is ethanol and the reagents comprise alcohol dehydrogenase, nicotinamide adenine dinucleotide, NADH diaphorase and a tetrazolium dye.

17. A method for determining the concentration of an analyte in a fluid sample comprising:
applying a fluid sample from an absorbent member of a sample collector to a device comprising
a test matrix comprising reagents for detecting the analyte; and
a sample application port for receiving a liquid sample, the port being in fluid communication with the test matrix;
a window through which at least a portion of the matrix is visible;
a standards card bearing standards and moveably connected to the device and having one or more color standards that correlate with an analyte concentration;
moving the standards card to a position so that the standard is adjacent to the test matrix; and
comparing the color of the test matrix to a standard to determine the concentration of analyte in the fluid sample.

18. The method of claim 17 wherein the fluid sample is saliva.

19. The method of claim 17 wherein the matrix changes from a first color to a second color when the reagents react with the analyte, and the second color is indicative of the concentration of the analyte.

20. The method of claim 17 wherein the step of moving the standards card comprises sliding the standards card linearly through the device so that the standard is adjacent to the test matrix.

21. The method of claim 17 wherein the step of applying the sample fluid comprises pressing the absorbent member of the sample collector against an expression surface on the device, the expression surface being located in the sample application port.

22. The method of claim 17 wherein the analyte is a drug of abuse.

23. The method of claim 17 wherein the analyte is ethanol.

24. A kit for detecting an analyte in a fluid sample, comprising packaged together:
a test device comprising
a sample collector for collecting and applying a liquid sample;
a test matrix comprising reagents for detecting the analyte;
a sample application port for receiving a liquid sample, the port being in fluid communication with the test matrix;
a storage port for storing the sample collector in contact with the device;
a window though which at least a portion of the matrix is visible; and
a standards card having one or more color standards for comparison with the test matrix; and
instructions for use of the device.

25. The kit of claim 24 wherein the application port further comprises an expression surface for separating the sample from the sample applicator.

26. The kit of claim 24 wherein the instructions describe the use of the device to detect ethanol in saliva.
